(19) United States
(12) Patent Application Publication
(10) Pub. No.: US 2018/0134788 A1
(43) Pub. Date: May 17, 2018

(54) Methods and Pharmaceutical Compositions (NTSR1 Inhibitors) for the Treatment of Hepatocellular Carcinomas

(71) Applicants: INSERM (Institut National de la Sante et de la Recherche Medicale), Paris (FR); Universite Paris Descartes, Paris (FR)

(72) Inventors: Patricia Forgez, Paris cedex 06 (FR); Zherui Wu, Paris cedex 06 (FR)

(73) Assignees: INSERM (Institut National de la Sante et de la Recherche Medicale), Paris (FR); Universite Paris Descartes, Paris (FR)

(21) Appl. No.: 15/574,288

(22) PCT Filed: May 26, 2016
(86) PCT No.: PCT/EP2016/061909

§ 371 Date: Nov. 15, 2017

(30) Foreign Application Priority Data
May 26, 2015 (EP) 15305784.9

(51) Int. Cl. C07K 16/28; A61P 35/00; C07K 16/30; A61K 47/68; C12N 15/113

(52) U.S. Cl. CPC C07K 16/286; A61P 35/00; C07K 16/303; A61K 47/6849; C12N 15/1138; A61K 2039/505; C07K 2317/24; C07K 2317/21; C07K 2317/732; C12N 2310/14; C07K 2317/76

(57) ABSTRACT

The present disclosure relates to methods and pharmaceutical compositions for the treatment of hepatocellular carcinomas (HCC in abbreviation, the term “hepatocellular carcinoma” has its general meaning in the art and refers to the cancer developed in hepatocytes see page 2 lines 24-30 of the present application) comprising administering to the subject a therapeutically effective amount of an inhibitor of NTSR1 (Neurotensin Receptor 1) activation or expression. The examples show the expression of NTSR1 in HCC cell lines and the role of the NTS/NTSR1 complex in these cell lines, thereby showing the role of the complex in tumour progression.

### FIELD OF THE INVENTION

The present invention relates to methods and pharmaceutical compositions for the treatment of hepatocellular carcinomas (HCC).

### BACKGROUND OF THE INVENTION

Hepatocellular carcinoma (HCC) is the sixth most common cancer worldwide with over 500 000 people diagnosed each year. Due to the combination of late diagnosis and a lack of curative treatments, HCC has become the second leading cause of cancer-related death in men, and the sixth leading cause of cancer-related death in women (CIRC, http://www.iarc.fr/, 2012). At diagnosis, most patients have already reached metastatic stages, and are not eligible for tumor ablation. The most common treatment for late stage patients are transarterial embolization and the transcatheter arterial chemoembolization (TACE). Recently, the use of sorafenib, an oral multikinase inhibitor, was shown to prolong survival in patients with advanced HCC and well-compensated liver function. Nevertheless, globally chemotherapeutic agents show very limited effectiveness. The adverse clinical course of most HCC patients underscores much need for more efficacious chemotherapies and development of targeting strategies.

Neurotensin (NTS) is a 13 amino acid peptide present and biologically active in the central nervous system and in the periphery. In the periphery, neurotensin acts as an endocrine hormone involved in the postprandial regulation of the motor and hormonal functions of the gastrointestinal tract. After meals, especially those rich in fatty acids, neurotensin is released by the endocrine cells (N) of the intestinal mucosa into the portal vein and then metabolized by liver. Typical physiological functions for neurotensin include stimulation of pancreatic and biliary secretions, inhibition of small bowel and gastric motility, and facilitation of fatty acids translocation.

NTS actions are mediated by three subtypes of neurotensin receptors, two G protein coupled receptors, NTSR1 and NTSR2 exhibiting high (sub-nanomolar) and low (nanomolar) affinity for NTS, respectively, and NTSR3 or gp/95/sortilin a single transmembrane domain receptor.

The contribution of the high affinity neurotensin receptor (NTSR1) and its ligand neurotensin in cancer progression has been shown in lung, breast, colon, head and neck squamous cell carcinomas. Sustained stimulation of NTSR1 by NTS generates autocrine regulation of epidermal growth factor receptors (HERs), resulting in the enhancement of cell aggressiveness.

Previous studies have suggested a role of neurotensin in HCC. Neurotensin is expressed in the fetal human liver, and repressed in the normal adult liver. However neurotensin was found reexpressed in the HCC subtype, the fibrolamellar carcinoma. In fibrolamellar carcinomas, serum neurotensin concentration was correlated with the response of the chemotherapy, and the stage of the patients. Little is known regarding the expression of NTSR1 in normal liver and HCC. Very slight amounts of high affinity binding site were found in the rat liver, and no receptor could be detected by autoradiography in HCC.

### SUMMARY OF THE INVENTION

The present invention relates to methods and pharmaceutical compositions for the treatment of hepatocellular carcinomas (HCC). In particular, the present invention is defined by the claims.

### DETAILED DESCRIPTION OF THE INVENTION

One aspect of the present invention relates to a method of treating hepatocellular carcinoma in a subject in need thereof comprising administering to the subject a therapeutically effective amount of an inhibitor of NTSR1 activation or expression.

As used herein the term “hepatocellular carcinoma” has its general meaning in the art and refers to the cancer developed in hepatocytes. In general, liver cancer indicates hepatocellular carcinoma in large. HCC may be caused by an infectious agent such as hepatitis B virus (HBV, hereinafter may be referred to as HBV) or hepatitis C virus (HCV, hereinafter may be referred to as HCV). In some embodiments, HCC results from alcoholic steatohepatitis or non-alcoholic steatohepatitis (hereinafter may be abbreviated to as “NASH”). The term also includes digestive hepatic micro-metastasis.

In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of HCC. The methods of the invention contemplate any one or more of these aspects of treatment.

As used herein, the term “NTSR1” has its general meaning in the art and refers to neurotensin receptor 1 (Gene ID: 4923) which belongs to the large superfamily of G-protein coupled receptors. The natural ligand of NTSR1 is neurotensin (NTS).

As used herein, the term “inhibitor of NTSR1 activation or expression” should be understood broadly, this expression refers to agents down-regulating the expression of neurotensin or of neurotensin receptor 1, compounds that bind to neurotensin (NTS) or NTSR1 and inhibit the neurotensin activation of NTSR1, or a protease that can degrade NTS.

Examples of inhibitors of NTSR1 activation or expression may be selected from the group consisting of an agent down-regulating the expression of NTS or NTSR1, an antibody against NTS or a fragment thereof which binds to NTS, an antibody against the NTSR1 or a fragment thereof which binds to the NTSR1, and an antagonist of the NTSR1.

As used herein, the term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In some embodiments, the antibody is a “chimeric” antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

In some embodiments, the antibody is a humanized antibody. “Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

In some embodiments, the antibody is a human antibody. A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

In some embodiments, the antibody is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the antibody is an anti-NTSR1 monoclonal antibody-drug conjugate. An “anti-NTSR1 monoclonal antibody-drug conjugate” as used herein refers to an anti-NTSR1 monoclonal antibody according to the invention conjugated to a therapeutic agent. Such anti-NTSR1 monoclonal antibody-drug conjugates produce clinically beneficial effects on NTSR1-expressing tumor cells when administered to a subject. In typical embodiments, an anti-NTSR1 monoclonal antibody is conjugated to a cytotoxic agent, such that the resulting antibody-drug conjugate exerts a cytotoxic or cytostatic effect on a NTSR1-expressing tumor cell when taken up or internalized by the cell. Any cytotoxic agent well known by the skilled person may be used. In some embodiments, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Publication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In some embodiments, the anti-NTSR1 monoclonal antibody of the invention is used to induce antibody dependent cellular cytotoxicity (ADCC) or complement dependent cytotoxicity (CDC) against NTSR1-expressing cells. In some embodiments, the anti-NTSR1 antibody may be suitable for disturbing the expression of NTSR1 at the cell surface (e.g. by provoking internalization of NTSR1) so that cell migration, cell proliferation and tumour growth of tumor cells will be limited or inhibited. In some embodiments, an anti-NTSR1 monoclonal antibody of the invention is used to induce antibody dependent cellular cytotoxicity (ADCC). In ADCC, monoclonal antibodies bind to a target cell (e.g., cancer cell) and specific effector cells expressing receptors for the monoclonal antibody (e.g., NK cells, CD8+ T cells, monocytes, granulocytes) bind the monoclonal antibody/target cell complex resulting in target cell death. Accordingly, in some embodiments, an anti-NTSR1 monoclonal antibody comprising an Fc region with effector function is used to induce antibody dependent cellular cytotoxicity (ADCC) or complement dependent cytotoxicity (CDC) against a NTSR1-expressing cell. Methods for inducing ADCC generally include contacting the NTSR1-expressing cell with an effective amount an anti-NTSR1 monoclonal antibody comprising an Fc region having ADCC activity, wherein the contacting step is in the presence of a cytolytic immune effector cell expressing an Fc receptor having cytolytic activity. Immune effector cells expressing cytolytic Fc receptors (e.g., FcγRIIIα or CD16) include, for example, NK cells as well certain CD8+ T cells. Methods for inducing CDC generally include contacting the NTSR1-expressing cell with an effective amount an anti-NTSR1 monoclonal antibody comprising an Fc region having CDC activity, wherein the contacting step is in the presence of complement.

In some embodiments, the anti-NTSR1 antibody is monospecific, bispecific, trispecific, or of greater multispecificity. Multispecific antibodies, including bispecific and trispecific antibodies, useful for practicing the methods described herein are antibodies that immunospecifically bind to both NTSR1 and a second cell surface receptor or receptor complex that mediates ADCC, phagocytosis, and/or CDC, such as CD16/FcgRIII, CD64/FcgRI, killer inhibitory or activating receptors, or the complement control protein CD59. In a typical embodiment, the binding of the portion of the multispecific antibody to the second cell surface molecule or receptor complex enhances the effector functions of the anti-NTSR1 antibody. In some embodiment, the anti-NTSR1 antibody is a bispecific antibody. The term “bispecific antibody” has its general meaning in the art and refers to any molecule consisting of one binding site for a target antigen on tumor cells (i.e. a NTSR1 receptor) and a second binding side for an activating trigger molecule on an effector cell, such as CD3 on T-cells, CD16 (FcyRlll) on natural killer (NK) cells, monocytes and macrophages, CD89 (FcαRI) and CD64 (FcyRI) on neutrophils and monocytes/macrophages, and DEC-205 on dendritic cells. According to the invention, the bispecific antibody comprises a binding site for NTSR1. tApart from the specific recruitment of the preferred effector cell population, bispecific antibodies avoid competition with endogenous immunoglobulin G (IgG) when the selected binding site for the trigger molecule on the effector cell does not overlap with Fc-binding epitopes. In addition, the use of single-chain Fv fragments instead of full-length immunoglobulin prevents the molecules from binding to Fc-receptors on non-cytotoxic cells, such as FcγRII on platelets and B-cells, to Fc-receptors that do not activate cytotoxic cells, including FcyRlllb on polymorphonuclear leukocytes (PMN), and to inhibitory Fc-receptors, such as FcyRllb on monocytes/macrophages. Methods for making bispecific antibodies are known in the art. Traditional production of full-length bispecific antibodies is based on the coexpression of two immunoglobulin heavy chain-light chain pairs, where the two chains have different specificities (see, e.g., Milstein et al., 1983, Nature 305:537-39). Because of the random assortment of immunoglobulin heavy and light chains, these hybridomas (quadromas) produce a potential mixture of 10 different antibody molecules, of which only one has the correct bispecific structure. Similar procedures are disclosed in International Publication No. WO 93/08829, and in Traunecker et al., 1991, EMBO J. 10:3655-59. Other examples of bispecific antibodies include Bi-specific T-cell engagers (BiTEs) that are a class of artificial bispecific monoclonal antibodies. BiTEs are fusion proteins consisting of two single-chain variable fragments (scFvs) of different antibodies, or amino acid sequences from four different genes, on a single peptide chain of about 55 kilodaltons. One of the scFvs binds to tumor antigen (i.e. NTSR1) and the other generally to the effector cell (e.g. a T cell via the CD3 receptor. Other bispecific antibodies those described in WO2006064136. In particular the bispecific antibody is a Fab format described in WO2006064136 comprising one VH or VHH specific for NTSR1 and one VH or VHH specific for an effector cell.

An “inhibitor of gene expression” refers to a natural or synthetic compound that has a biological effect to inhibit or significantly reduce the expression of a gene.

Inhibitors of gene expression for use in the present invention may be based on anti-sense oligonucleotide constructs. Anti-sense oligonucleotides, including anti-sense RNA molecules and anti-sense DNA molecules, would act to directly block the translation of the mRNA by binding thereto and thus preventing protein translation or increasing mRNA degradation, thus decreasing the level of the protein (e.g. NTSR1), and thus activity, in a cell. For example, antisense oligonucleotides of at least about 15 bases and complementary to unique regions of the mRNA transcript sequence encoding the targeted protein (e.g. NTSR1) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting a subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the targeted proteins (e.g. NTSR1). Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

By a “therapeutically effective amount” is meant a sufficient amount of the inhibitor of NTSR1 activity or expression at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitor of NTSR1 activity or expression is typically combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: HCC immune-staining for NTSR1 and NTS long fragment.

Tissues Micro Arrays from two diagnosis centers were assayed for NSTR1 and NTS LF labelling. Examples for negative and positive labelling for NTSR1 (Top) and NTS LF (Bottom) are shown.

FIG. 2: Overall survival of patients with HCC (n=368) according to mRNA NTSR1 expression Overall survival according to the expression of NTSR1.

Shown is the Kaplan-Meier plot according to (A) NTS mRNA (B) NTSR1 expression (C) both levels over (solid line) or below (broken line) the 90th percentile.

FIG. 3: Expression of NTS and NTSR1 in HCC cell lines

(A) NTS, NTSR2, NTSR3, and NTSR1 transcript analysis from 400 ng of HuH7, HEP 3B, and PLC/RF5, total RNA. NTSR1 A and NTSR1 B show the amplicon from two different set of primers. (B) Typical immunocytochemistry labeling for NTSR1 and DAPI in HEP 3B and PLC/RF5 cells. Cells were seeded on glass slides, grown for 48 h, and immunocytochemistry was performed as described in the Materials and Methods section. (C) NTSR1 transcript analysis from HuH7, HEP 3B and PLC/RF5 of total RNA from cells treated or not with 20 nM LiCl for 6 h. (D) Typical immunocytochemistry NTSR1 labeling of HEP 3B and PLC/RF5 cells treated with 1 μM SB 216763 for 6 h.

FIG. 4: EGFR expression and activation by NTS/NTSR1 complex in HCC cell lines.

(A) Typical immunocytochemistry labeling for NTSR1 (Top) and EGFR (Bottom) in PLC/RF5 and HEP 3B cells treated or not with 1 μM SB216763 or 5 μM SR4892 or both for 48 h. (B and C) Representative western blot analyses of Tyr 1173 phosphorylated EGFR, EGFR, and actin of HEP 3B and PLC/RF5 treated as indicate above.

FIG. 5: EGFR expression and activation by NTS/NTSR1 complex in HCC cell lines overexpressing NTSR1.

(A) NTSR1 transcript analysis from HEP 3B and PLC/RF5 wild type and two chosen clones stably transfected with NTSR1 coding sequence for each cell line. Two set of primers were tested for NTSR1. (B and C) Semi quantitative calculation of EGFR and phosphorylated EGFR protein content from HEP 3B and PLC/RF5 wild type cells and clone overexpressing NTSR1. Results from 4 experiments are expressed as relative to respective wild type cells.

Inset: Representative western blot analyses of EGFR, and Tyr 1173 phosphorylated EGFR of HEP 3B and PLC/RF5 and clones.

FIG. 6: NTS autocrine regulation enhanced migration speed and invasion on type 1 collagen matrix.

(A) Speed of migration on type 1 collagen of HEP 3B and NTSR1 overexpressing clones treated or not with 1/100 NTS or 1/100 Long Fragment NTS antibody. Results represent the mean±SEM of 5 independent experiments. (B) Speed of migration on type 1 collagen of PLC/RF5 and NTSR1 overexpressing clones in the presence or not of 5 μM SR 48692. Results represent the mean±SEM of 3 independent experiments. (C) Numbers of invasive cells crossing the type I collagen in Boyden chamber when HEP 3B and NTSR1 overexpressing clones were treated or not with NTS or Long Fragment NTS antibody or 5 μM. SR 48692. Results are expressed relatively to wild type cells and represent the mean±SEM of 5 to 7 independent experiments.

FIG. 7: NTS/NTSR1 complex enhanced experimental tumor growth in HCC cells lines.

(A) Experimental tumors were generated from the HCC cancer cell line, HEP 3B and PLC/RF5 and the NTSR1-overexpressing subclones. Comparative growth curves of HEP 3B, HEP-R1a, PLC/RF5, PLC/R1 in 8, 7, 10 and 7 mice, respectively. Tumor volumes were measured twice a week. (B) Tumor weight at 42 days. (C and D) Experimental tumors were generated from HEP 3B or HEP-R1a in the different mice. When the tumor size reached 100 mm3, mice were randomly distributed in 2 groups. A control group force feed with H2O, or with 1 mg/kg SR 48692 every day. (E) Typical IHC performed on paraffin sections for NTS, NTSR1 EGFR and phosphorylated EGFR labelling, 200× magnification.

FIG. 8: Neurotensin restores the response to erlotinib or sorafenib in vivo.

Experimental tumors were generated from HEP 3B (A) and HEP-R1a (B) in the same mice. Mice were treated per os with 75 mg/kg erlotinib every day for 22 days. Tumor size was measured every two days. Result shown is the ratio of tumor compare to day 1. (C) The doubling time was subsequently calculated using the following formula: doubling time=t*((LN(2))/(LN(VOL F/VOL I)), at day 22.

Experimental tumors were generated from HEP 3B (D) or HEP-R1a (E) in the different mice. When the tumor size reached 100 mm³, mice were randomly distributed in 2 groups. A control group force feed with H₂O, and the treated group, force feed every day with 15 mg/kg sorafenib. Result shown is the ratio of tumor compare to day 1. (F) Tumor weight at day 15.

FIG. 9: Neurotensin regulation restores response to erlotinib and sorafenib in vitro.

(A) Cellular proliferation performed on HEP 3B and HEP-R1b cells. Results are the means of 3 independents experiments and expressed as percentage of respective control. (B) Clonogenic assay performed with HEP 3B and HEP-R1b cells treated with different dose of erlotinib. Cells were incubated for two weeks with the treatment. Results are the means of 6 independents experiments and expressed as percentage of respective control. (C) Cellular proliferation performed on HEP 3B and HEP-R1a cells. Results are the means of 6 independents experiments. (D) Clonogenic assay performed with HEP 3B and HEP-R1a cells treated with different dose of sorafenib. Cells were incubated for two weeks with the treatment. Results are the means of 6 independents experiments.

### EXAMPLE

NTS and NTSR1 are Expressed in HCC

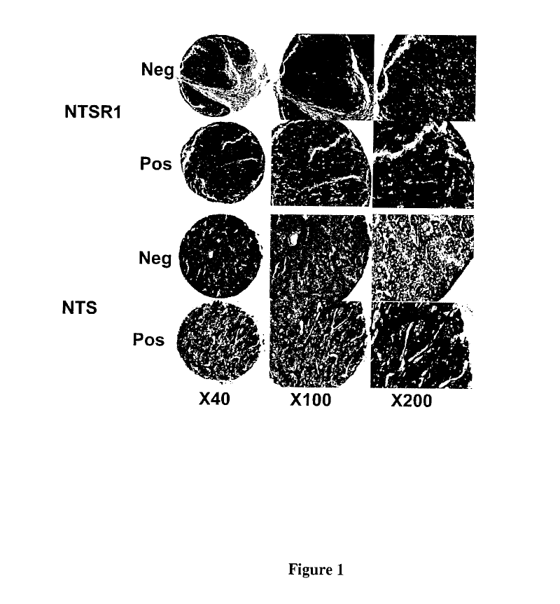

Expression of NTS and NTSR1 was scored on TMA containing tumors of patients with HCC. NTSR1 labelling was seen on 40/72 (56%) of tumors where the staining was clonal, and more intense in the front of the tumor and sometimes polarized. FIG. 1 (top) shows an example of NTSR1 negative and positive labelling. We studied NTS long fragment labelling, in order to evaluate if tumor cells synthesize NTS. Amongst the specimens, 47/73 (64%) were scored as positive. FIG. 1 (bottom) shows an example of a positive and negative labeling. Autocrine NTS regulation, as evaluated on successive slides was seen in 31/63 (49%) tumors. These observations suggest a potential contribution of NTS/NTSR1 complex in HCC carcinogenesis and progression. We further analyzed the situation with in vitro and in vivo studies to clarify this contribution.

High NTS or NTSR1 mRNA Expression is Correlated with Worse Overall Survival in the HCC Patients.

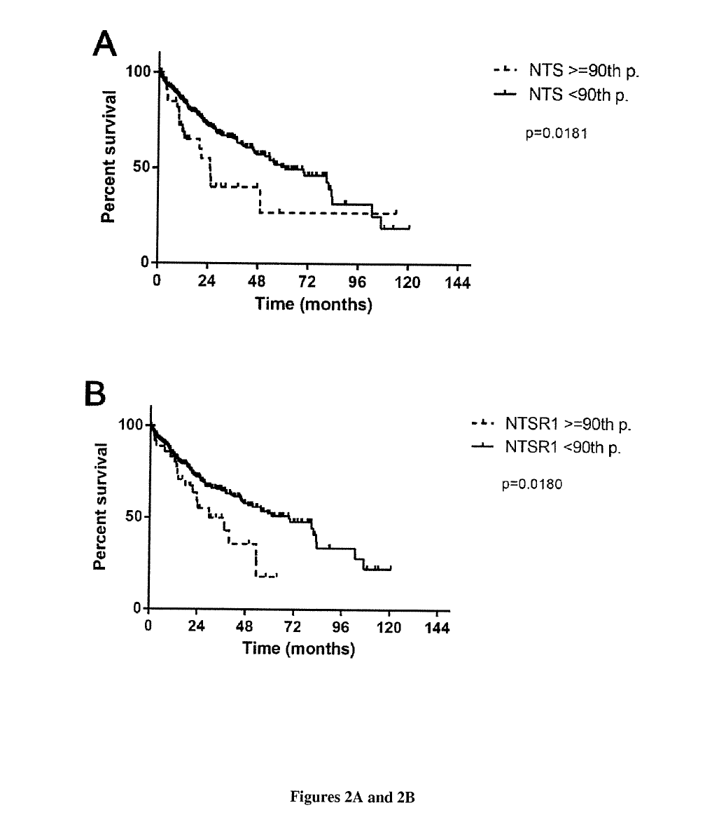

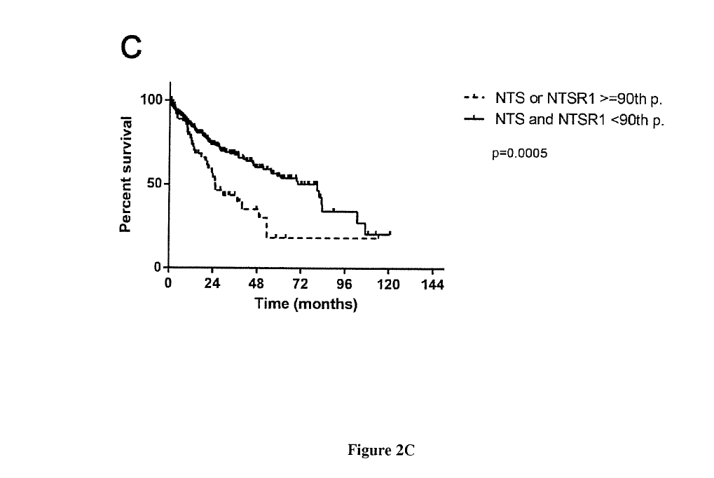

The TCGA database includes a cohort of 367 HCC cases with both NTS and NTSR1 mRNA expression data analyzed by Illumina Genome Analyzer RNA sequencing. Kaplan-Meier analysis of the overall survival (OS) for this dataset showed that a high NTS mRNA expression (expression z-score above the 90 th percentile) was associated with a significantly worst OS (Log-rank test P=0.0181). The 5-year survival rates for patients with high NTS expression was 26.67% compared with 49.50% for patients with low NTS expression, and the median survival for these patients was 25.13 and 60.84 months (FIG. 2 A).

Similar finding was found using NTSR1 mRNA expression. The OS was significantly worst in patients with high NTSR1 mRNA expression (expression z-score above the 90 th percentile) compared to those with low level of NTSR1 mRNA (Log-rank test P=0.0180). The 5-year survival rates was 17.87% and 51.08%, respectively, their median survival was 31.29 and 69.51 months, respectively (FIG. 2 B).

In the same dataset, when we compared the patients with high mRNA expression for at least one of the targets NTS and NTSR1 to the patients with low expression for both NTS and NTSR1. A significantly better OS was found in patients with moderate or low mRNA expression for both NTS and NTSR1 (Log-rank test P=0.0005), the 5-year survival rates was 53.80% for them compared with 18.07% in patients with high expression for NTS and NTSR1, the median survival for these patients was 80.68 and 25.13 months, respectively (FIG. 2 C).

NTS and NTSR Expression in HCC Cells Lines

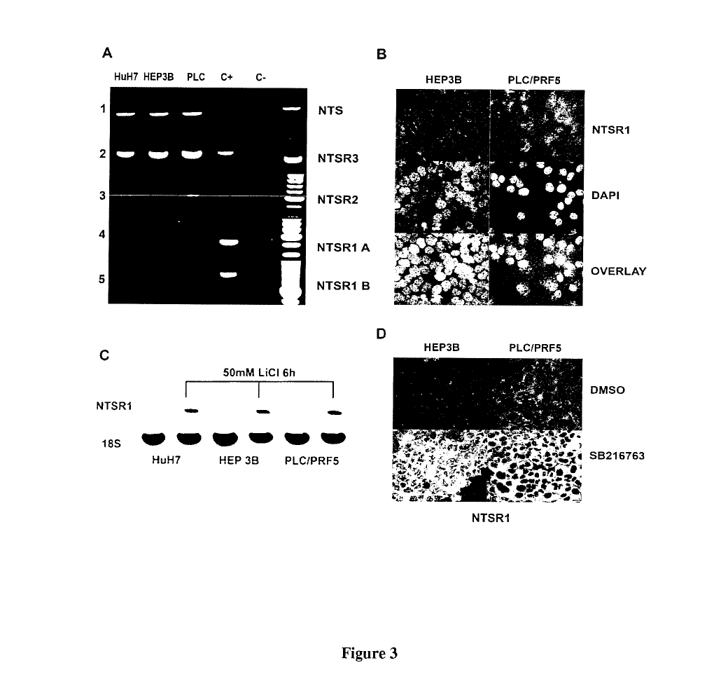

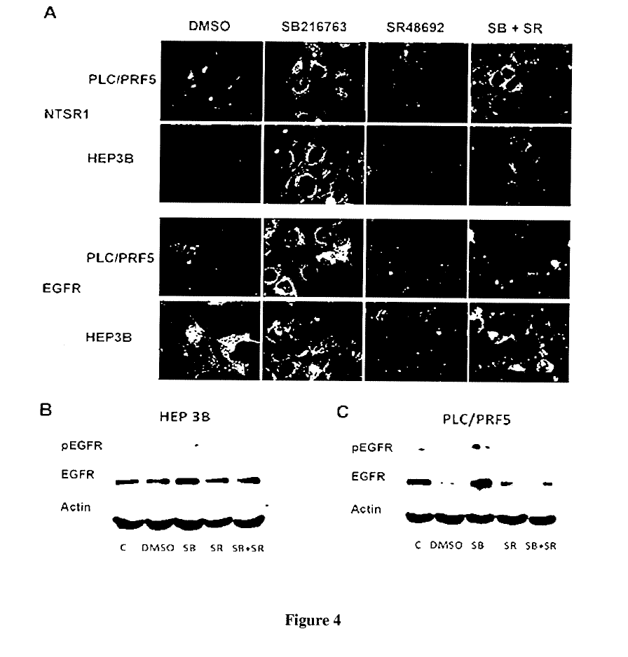

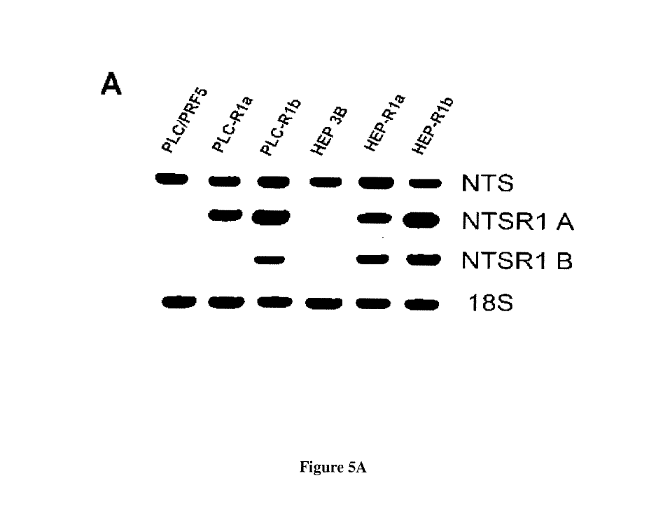

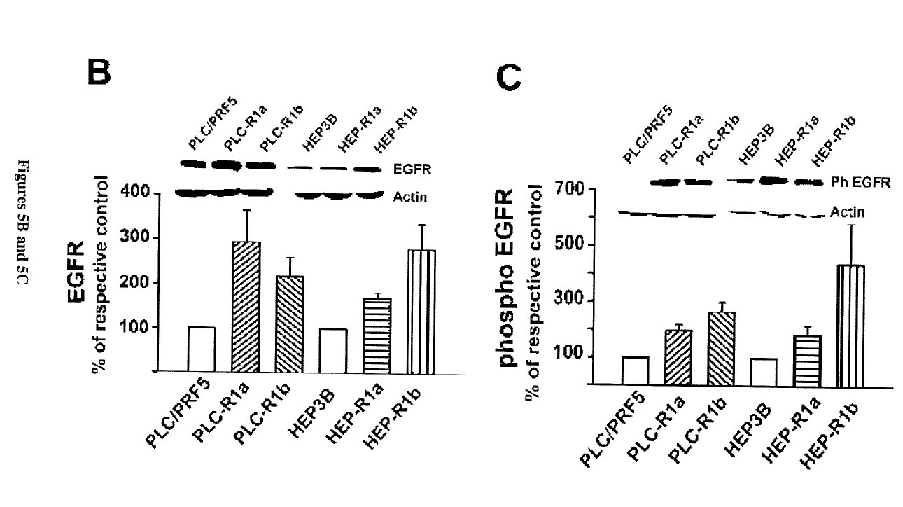

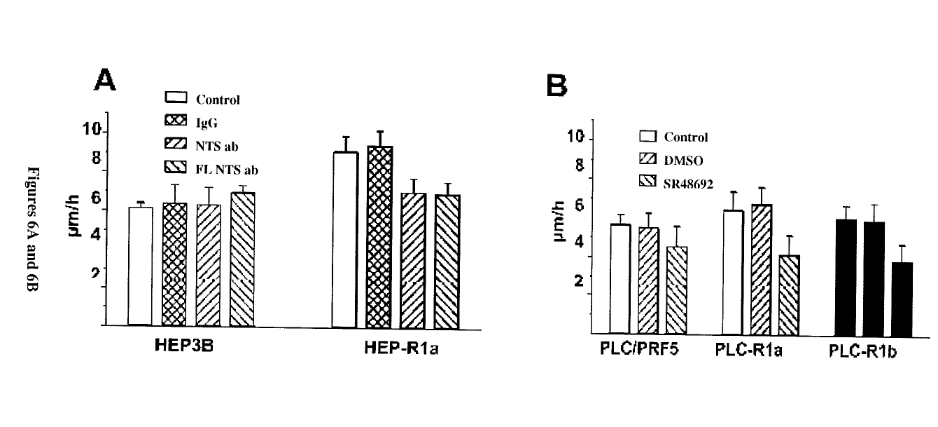

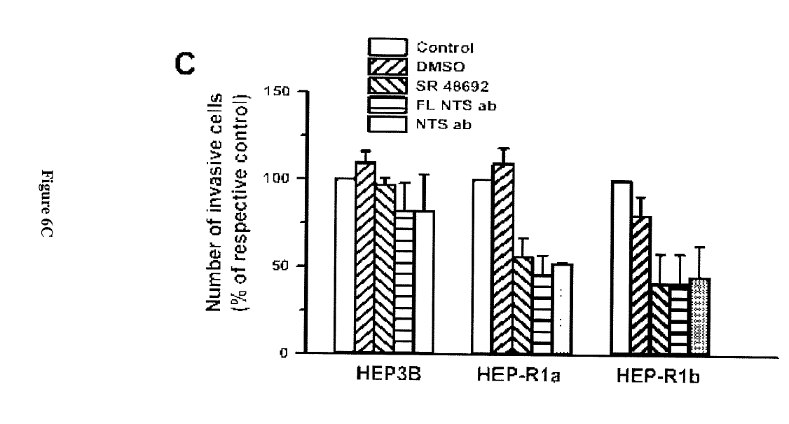

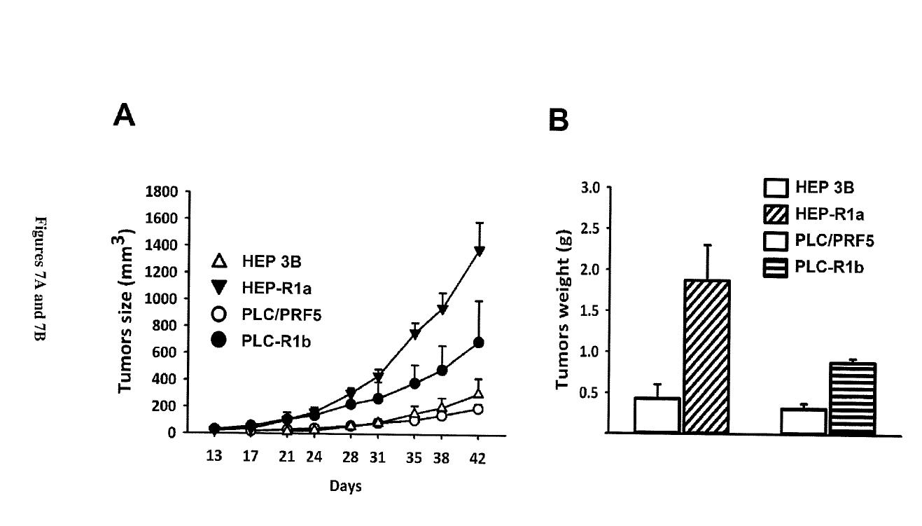

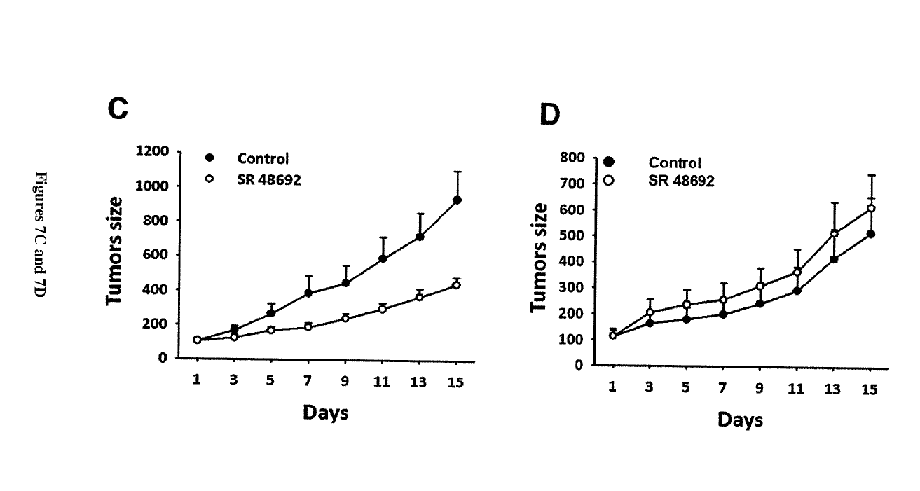

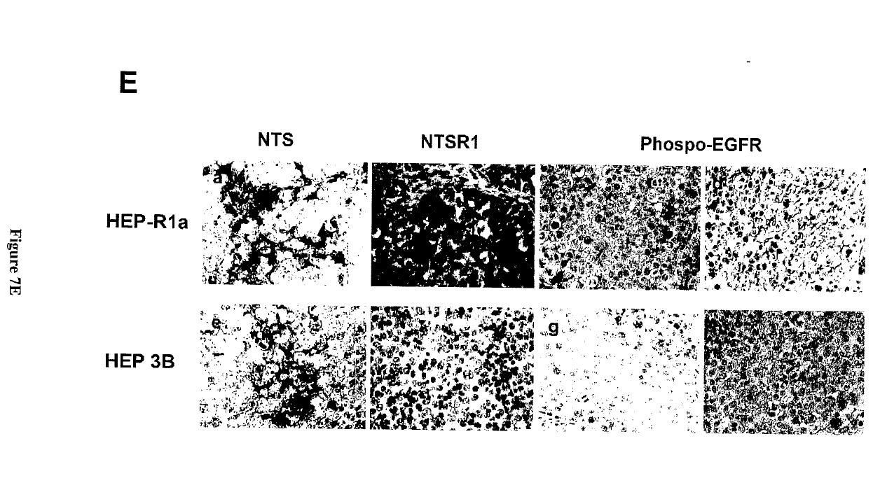

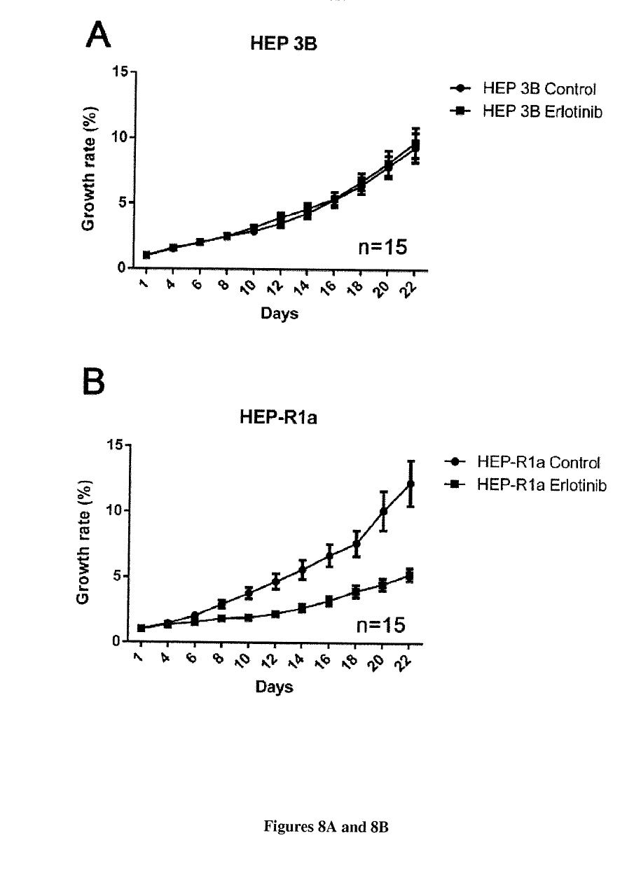

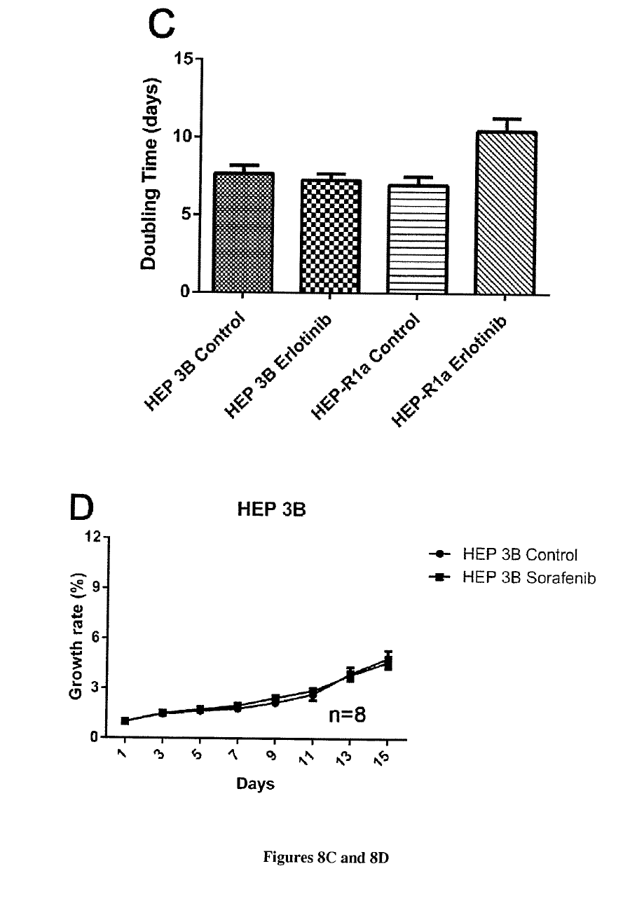

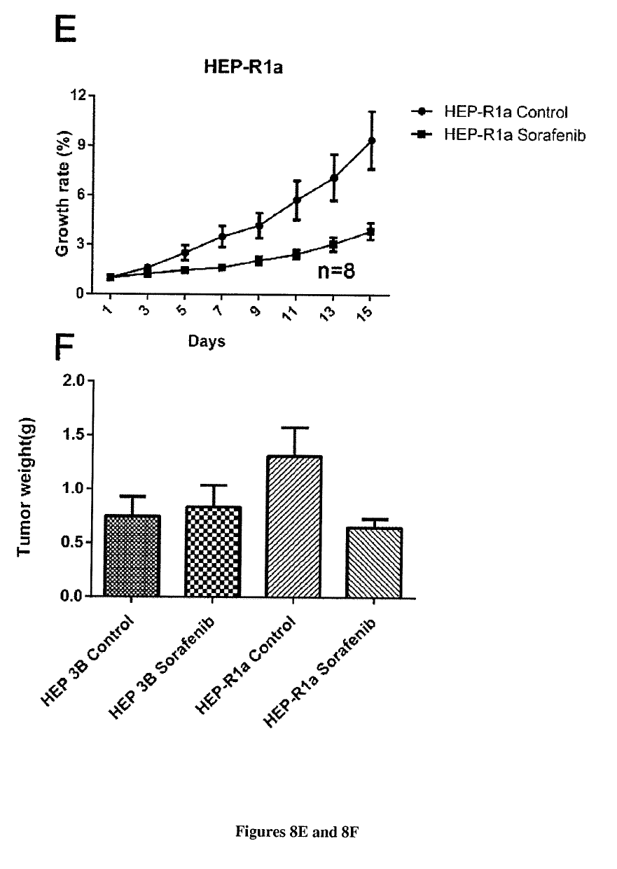

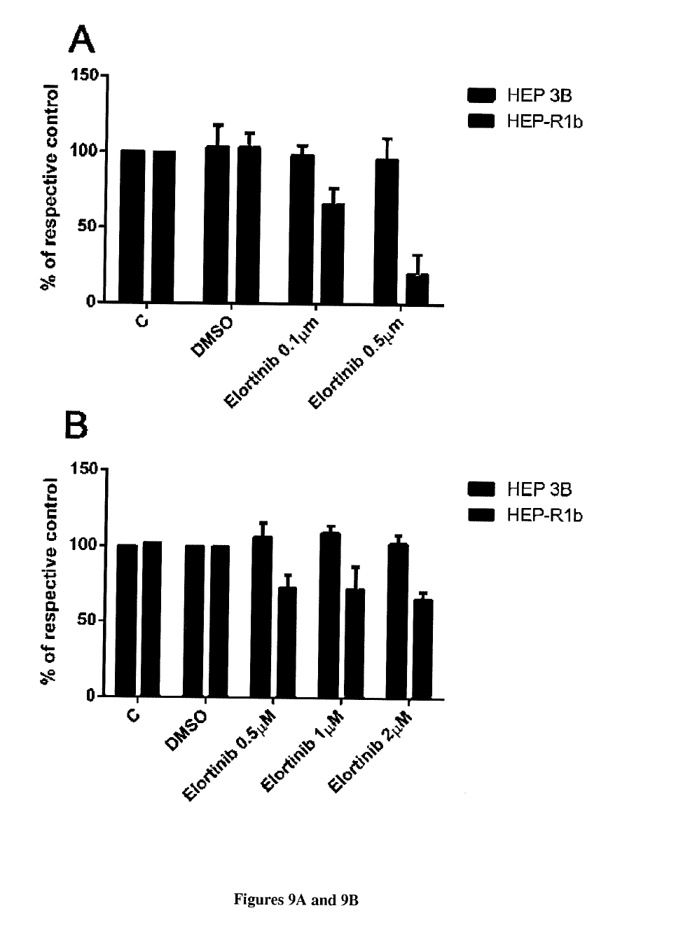

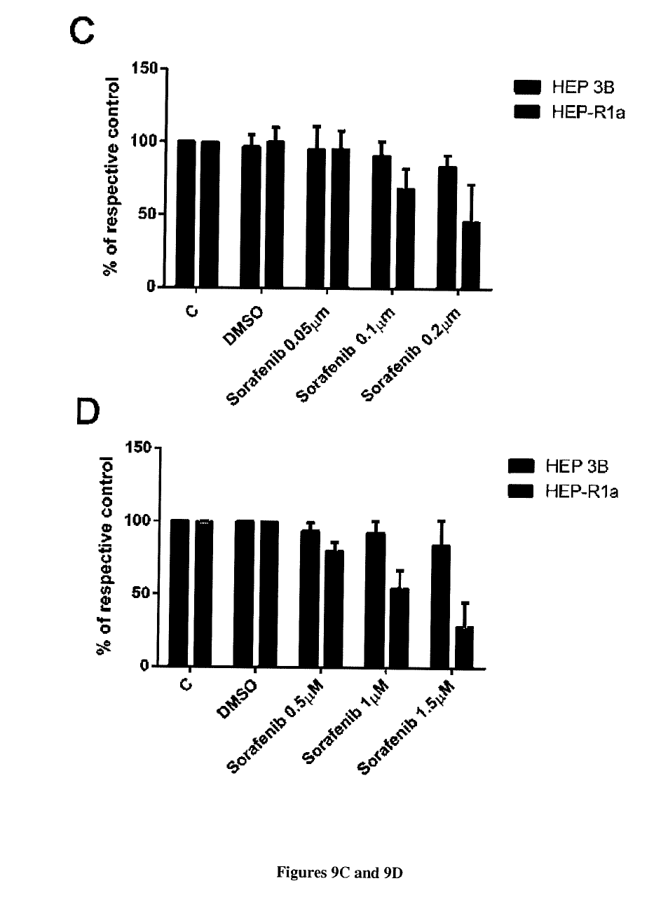

We searched for NTS and the NTS receptors mRNA expression in three HCC cell lines, PLC/PRF5, HEP 3B, HuH7. As shown in FIG. 3A, NTS, NTSR2, and NTSR3 are expressed in the three cell lines, lane 1, 2, and 3, respectively. NTSR1 mRNA was only detected in PLC/PRF5 cells (FIG. 3A lane 4). The presence of NTSR1 transcript was confirmed with a second set of primer (FIG. 3A lane 5). The NTSR1 cellular protein content was visualized by immunofluorescence assay. Intracellular cluster of NTSR1 fluorescent labelling was detected only in PLC/PRF5 cells. This labeling was reinforced close to the nucleus, suggesting an activated NTSR1.

NTSR1 Expression in HCC Cells is Regulated by Wnt/Beta-Catenin Pathway

Among the three cell lines studied, only PLC/PRF5 cells presented a dysregulation in the beta catenin pathway with a deletion in Axin (E4/−). We previously showed that the NTSR1 gene is a target for the Tcf-beta/catenin complex, conducting an abnormal expression of NTSR1 in the early stage of colon cancer. We previously hypothesized that NTSR1 expression was correlated with deregulated wnt/beta catenin pathways. To evaluate this hypothesis, we forced the cellular and nuclear relocation of β-catenin in these cells lines with the LiCl, and an inhibitor of the glycogen synthase kinase (GSK) 3β, the SB216763. LiCl and SB216763 are known to decrease the degradation of β-catenin by inhibiting GSK-3 and promoting the accumulation of β-catenin in the cytoplasm of liver cancer cells. As shown in the FIG. 2 C NTSR1 transcription was upregulated in the three HCC cell lines after LiCl exposure. The same observation was made when cells were treated with SB216763, the NTSR1 immunolabeling signal was remarkably amplified in HEP3B and PLC/PRF5 cells (FIG. 4 D), suggesting a correlation between NTSR1 expression in HCC and the activation of Wnt/β-catenin pathway.

NTS/NTSR1 Complex Induced Expression and Activation of EGFR

It was previously shown that the NTS/NTSR1 complex potentiates EGF signaling in liver, colon, prostate, lung, and breast cancer. We investigated the possible relation between NTSR1 expression and EGFR expression and activation. As shown in FIG. 5A, the treatment with SB216763 for 48 hours induced an increase of NTSR1 and EGFR expression in both cell lines PLC/PRF5 and HEP3B. Treatment with SR48692, a specific NTSR1 antagonist, decreased the expression of EGFR, whereas NTSR1 expression was not altered. When the combined treatment with SB216763 and SR48692 is used, the effect of SB216763 on EFGR expression is limited by the presence of the NTSR1 antagonist (FIG. 5A).

The activated form of EGFR was evaluated by western blots, as shown in the FIGS. 5 B and C. In both cell lines, phosphorylated-EGFR was increased under SB216763 treatment this increase was abolished when SR 48692 was added to the treatment. This result suggests that NTS/NTSR1 complex upregulate EGFR and its activation in HCC cells.

In order to avoid the use of chemicals to induce an increase of NTSR1 expression, the HCC cell lines HEP 3B and PLC/PRF5 were transfected with the plasmid pcDNA3 coding for NTSR1. Two stable clones were selected for each cell line, one clone moderately expressed NTSR1, PLC-R1a, and HEP-R1a, and one clone overexpressed NTSR1, PLC-R1b, and HEP3b (FIG. 5A two NTRS1 primer sets were tested). Compared to the parental cells, the total EGFR expression was up regulated in all clones generated in PLC/PRF5 or HEP3B cells (FIG. 5B). The activation of EGFR was also studied by examining the phosphorylation of the tyrosine 1173, one of the major sites of auto phosphorylation on the C-terminal on EGFR. In all clones, the basic level of phosphorylated EGFR is raised by 2 to 4 fold when generated from PLC/PRF5 cells and 2 fold when generated from HEP 3B, (FIG. 5C).

NTS/NTSR1 Complex Enhances Migration and Invasion of HCC Cells

Migration assays were used to evaluate the effect of NTS/NTSR1 on cancer cell migration, another hallmark contributing to the invasion and metastasis of cancers. The procedure described in the methods section allows the comparison of the migration speed between both cell lines at the same time over a period of 48 h, and on the intact type I collagen matrix. As shown in FIG. 6, HEP-R1a cells migrate at the speed of 9.1±0.78 μm/h, whereas HEP 3B cells were slower with a migration speed at 6.13±0.27 μm/h (n=5, p=0.002). Addition of NTS or long fragment NTS antibodies in the cells culture media inhibited the acceleration of HEP-R1a cells, which return to a migration speed similar to wild type cells. As a control, the isotype-matched IgG didn't influence the migration speed (FIG. 6A). Similar observations were made with PLC/PRF5 cells and derivative clones. The respective migration speed were not significantly different between parental cells and clones, with 5.63±0.52, 6.44±0.93, and 6.03±0.66 μm/h for PLC/PRF5, PLC-R1a and PLC-R2a, respectively. When cells were exposed to a specific NTSR1 antagonist, SR48692, the speed of PLC-R1a and PLC-R2a cells significantly decrease by 27% (p=0.02 vs DMSO) and 33% (p=0.01 vs DMSO), respectively. Overexpressing NTSR1 in PLC/RF5 did not enhance the migration speed (FIG. 6B).

In parallel, Boyden chambers were used to examine the invasiveness characteristics of HCC cancer cells, on type-I collagen-coated inserts over 36 hours incubation. Compared with HEP 3B cells, the number of invading cancer cells for HEP-R1a cells and HEP-R1b cells is increased by 211±19% (p<0.01) and 617±94% (p<0.01), respectively. To determine whether NTS contributes to cellular invasion, cells were exposed to the NTSR1 antagonist, or two antibodies directed against NTS or the long fragment NTS. The three components fully or partially abolished the increase of cellular invasion observed when NTSR1 is upregulated in HEP3B cells (FIG. 6C). In our hands, the PLC/PRF5 cells were very invasive. The over expression of NTSR1 did not markedly change these characteristics. Collectively, these data suggest that the NTS and NTSR1 promote the invasiveness and migration of HCC cells.

NTS/NTSR1 Enhance Experimental HCC Tumor Progression

To determine the contribution of NTS/NTSR1 complex on tumor progression, mice were implanted with HCC cell lines and NTSR1 overexpressing clones. For HEP3B cells the tumors were measurable 21 days after cells injection. In contrast, the tumor burden for both clones overexpressing NTSR1 was measurable at day 13. The growth rate at day 42 was 2.9 and 2.08 fold higher for HEP-R1a and PLC-R1a as compared to respective parental cells, respectively, (FIG. 7A). As shown in FIG. 7B the tumor weights were in correspondence with the tumor size 4.3 and 2.8 fold higher for HEP-R1a and PLC-R1a as compared to their respective parental cells, respectively. To confirm the contribution of NTS/NSTR1 complex in tumor growth, animals were treated with a specific NTSR1 antagonist, the SR 48692. When tumors reach 100 mm3, the groups were treated per os with H₂O, or 1 mg/kg SR 48692, respectively. SR 48692 treatment has no influence on the HEP 3B tumor growth rate, but inhibited by two fold the growth rate of HEP-R1a tumors (FIGS. 7 C and D). The final tumor size of HEP-R1a tumors treated with SR 48692 was 441.9±39.85 mm³, and was very similar to the untreated HEP 3B tumors 516.3±139.72 mm³.

We confirmed the expression of NTS in the tumors of HEP 3B and HEP-R1a cells with an antibody directed against the long fragment NTS. In both tumors, clusters of strong intra-cytoplasmic labelling were distributed randomly on the slide (FIG. 7E a and e). The labelling of NTSR1 in HEP-R1a revealed to be mostly intra-cytoplasmic and weak, with a few exceptional clusters of cells with very strong cytoplasmic and membrane expression (FIG. 7E b). As expected, no NTSR1 labelling was seen in HEP3B tumors (FIG. 7E F). In HEP-R1a tumors the labelling intensity of phosphorylated EGFR was heterogeneous and very strong with a thick to a thin line around the entire cell membrane. The strong labeling was often localized at the front of the tumor or close to the blood vessel. Examples of a thin or strong labelling are shown in FIGS. 7E C and D, respectively. In contrast, in HEP3B tumors, phosphorylated EGFR labeling was absent or very weak as shown in FIGS. 7E G and H.

NTS/NTSR1 Restores Responses to Tyrosine Kinase Inhibitors.

As shown above, NTSR1 activation induced a sustained EGFR activation, which we believe, acts as EGFR driver mutation (FIG. 4). To explore this hypothesis, the HCC cell lines HEP 3B and HEP-R1a were xenografted on the same mice. The mice were randomly distributed in two groups based on the size of the HEP 3B tumors. Since HEP-R1a tumors grow faster than HEP 3B tumors, HEP 3B cells were injected a few days before HEP-R1a cells. In the control group, the average HEP3B tumor size was 168.2±38.1 mm3 and the HEP-R1a tumor size was 79.7±16.4 mm3 at day 1. The treated group carried HEP 3B tumors of 164.7±31.2 mm3 and HEP-R1a tumors of 87.2±19.13 mm3 at day 1. Mice were daily treated, per os, with 75 mg/kg erlotinib or H₂O for 22 days. The growth rate of the HEP-R1a tumor was drastically affected by the EGFR inhibitor, whereas HEP3B tumors did not respond to erlotinib (FIGS. 8 A and B). Over the period of the treatment, the doubling time of HEP-R1a tumors was 7.0±0.55 days and 10.1±1.1 (p=0.0016) for control and treated with erlotinib, respectively. For the HEP 3B tumors, the doubling time was similar 7.8±0.52 and 7.7±0.84 days for control and treated group respectively (FIG. 8 C). The contribution of NTS/NTSR1 complex to tumor growth is relayed by EGFR activation, suggesting that tumor over expressing NTSR1 may be responsive to EGFR inhibitor.

Sorafenib is a known multikinase inhibitor targeting Raf/MEK/ERK signaling at the level of Raf kinase, angiogenesis, VEGFR-2/-3, and PDGFR-β tyrosine kinase. Sorafenib is currently used to treat HCC with some success. In order to evaluate the autocrine NTS regulation on sorafenib responses, each HEP 3B or HEP-R1a cells were injected to four groups of mice. When tumors reach 100 mm3, the groups were treated per os with H2O, 30 mg/kg sorafenib for 15 days, respectively. As the treatment has no influence on the HEP 3B tumor growth rate, sorafenib inhibited by two fold the growth rate of HEP-R1a tumors (FIGS. 8 D and E). Actually, the final tumor size of HEP-R1a tumors treated with sorafenib was 400.3±59.9 and was very similar to the untreated HEP 3B tumors 516.3±139.72 mm3. The tumors were weighed after the end of treatment. For the HEP 3B tumors, the tumor weight was approximately equal for the control and sorafenib treated group, On the other hand, for HEP-R1a tumors, sorafenib reduced tumor weight from 1.313±0.264 g in control group to 0.650±0.080 g in treated group (FIG. 8 F).

Cells Expressing NTS/NTSR1 are Sensitive to Tyrosine Kinase Inhibitors.

We confirmed the erlotinib and sorafenib responses in cells overexpressing NTSR1 in clonogenic assay. As shown in FIGS. 9 A and 9 C, the number of colonies made by HEP-R1b and HEP-R1a cells were markedly reduced when cells were exposed to erlotinib or sorafenib, respectively. In contrast, HEP 3B cells are not sensitive either to erlotinib or sorafenib. In the same vein, cellular proliferation test provide the same result, the cells overexpressing NTSR1 are more sensitive to erlotinib and sorafenib than HEP 3B cells (FIGS. 9 B and D).

### REFERENCES

Throughout this application, various references describe the state of the art to which this invention pertains. The disclosures of these references are hereby incorporated by reference into the present disclosure.

1. A method of treating hepatocellular carcinoma (HCC) in a subject in need thereof comprising administering to the subject a therapeutically effective amount of an inhibitor of NTSR1 activation or expression.

2. The method of claim 1 wherein the HCC results from alcoholic steatohepatitis, non-alcoholic steatohepatitis or is caused by an infectious agent such as hepatitis B virus or hepatitis C virus.

3. The method of claim 1 wherein the HCC is early stage HCC, non-metastatic HCC, primary HCC, advanced HCC, locally advanced HCC, metastatic HCC, HCC in remission, or recurrent HCC.

4. The method of claim 1 wherein the inhibitor of NTSR1 activation is selected from the group consisting of an agent down-regulating the expression of NTS or NTSR1, an antibody against NTS or a fragment thereof which binds to NTS, an antibody against the NTSR1 or a fragment thereof which binds to the NTSR1, and an antagonist of the NTSR1.

5. The method of claim 1 wherein the inhibitor of NTSR1 activation is an antibody against NTSR1.

6. The method of claim 5 wherein the antibody is a chimeric antibody, a humanized antibody or a human antibody.

7. The method of claim 5 wherein the antibody is an anti-NTSR1 monoclonal antibody-drug conjugate.

8. The method of claim 1 wherein the anti-NTSR1 monoclonal antibody is used to induce antibody dependent cellular cytotoxicity.

9. The method of claim 1 wherein the inhibitor of NTSR1 expression is a siRNA.
